(19) United States
(12) Patent Application Publication
(10) Pub. No.: US 2026/0263374 A1
(43) Pub. Date: Sep. 10, 2026

(54) COMPOSITIONS OF NANOPARTICLES FOR TREATMENT OF NEUROPATHIC PAIN

(71) Applicant: NANOBIOTIX S.A., PARIS (FR)

(72) Inventors: MARIE-EDITH MEYRE, PARIS (FR); FRÉDÉRIC SEDEL, PARIS (FR); CÉLINE BERJAUD, PARIS (FR)

(21) Appl. No.: 18/998,167

(22) PCT Filed: Jul. 28, 2023
(86) PCT No.: PCT/EP2023/070958

§ 371 Date: Jan. 24, 2025

(30) Foreign Application Priority Data
Jul. 28, 2022 (EP) 22306126.8

(51) Int. Cl. A61K 9/51; A61K 9/00; A61K 33/242; A61P 25/02; B82Y 5/00

(52) U.S. Cl. CPC A61K 9/5146; A61K 9/0019; A61K 9/5138; A61K 33/242; A61P 25/02; B82Y 5/00

(57) ABSTRACT

The invention concerns a novel and innovative composition for the treatment of neuropathic pain (NP). Specifically, the invention concerns nanoparticles and/or aggregates of nanoparticles, a composition comprising said nanoparticles and/or aggregates of nanoparticles, and their use in the treatment of NP.

### TECHNICAL DOMAIN

The invention concerns a novel and innovative composition for the treatment of neuropathic pain (NP). Specifically, the invention concerns nanoparticles and/or aggregates of nanoparticles, a composition comprising said nanoparticles and/or aggregates of nanoparticles, and their use in the treatment of NP.

### Preamble

Neuropathic pain (NP) is characterized by abnormal hypersensitivity to stimuli (hyperalgesia) and nociceptive responses to non-noxious stimuli (allodynia). These patients with hyperalgesia experience an increase in the perception of pain generated by a stimulus that causes pain, while patients with allodynia experience an increase in the perception of pain due to a stimulus that does not normally provoke pain. NP patients may also suffer from paresthesia (abnormal sensations comparable to needle bites, tingling, itching), or even loss of sensitivity. NP have continuous and/or episodic (paroxysmal) components. The latter resemble stabbings or electric shocks.

Many different conditions may lead to NP, for example, metabolic disorders, like diabetes, viral infections (for example, post herpetic neuralgia) and autoimmune diseases affecting the central (CNS) and peripheral nervous system (PNS), like multiple sclerosis and Guillain-Barré syndrome, respectively. Neuropathic pain is common in cancer patients as a direct result of cancer on peripheral nerves (e.g., compression by a tumor), radiation injury or surgery, or as a side effect of chemotherapy (chemotherapy-induced peripheral neuropathy, (CIPN)). For example, oxaliplatin, a platinum-based chemotherapy drug, is commonly used for the treatment of various types of cancer. Oxaliplatin is now approved in many countries as initial therapy for the treatment of colorectal cancer. The clinical value of oxaliplatin however, is reduced by acute and chronic forms of peripheral neuropathy including mechanical hyperalgesia that appear as side-effects in both humans and rodents. This oxaliplatin-induced peripheral neuropathy is the most frequent dose-limiting toxicity associated with the therapy, and currently no treatment for it is available.

Other causes of neuropathic pain include trauma-based damage to the nervous system like spinal cord injury (SCI), peripheral nerve injury including post-traumatic NP or post-surgical NP. Complications like neuroma following trauma and/or surgery may also induce NP.

Certain inflammatory disorders, hereditary neuropathies and channelopathies also exist [Colloca L, Ludman T, Bouhassira D, et al. (2017) Neuropathic pain. Nature Reviews Disease Primers 3:17002]. Neuropathic pain in the general population is estimated to have a prevalence ranging between 3% and 17% [Cavalli, E., et al., (2019) The neuropathic pain: An overview of the current treatment and future therapeutic approaches, Int. J. Immunopathology & Pharmacology, Vol. 33: 1-10].

Neuropathic pain can be associated with ectopic afferent discharges, which stem from a higher excitability and a repetitive firing capability of the affected neurons/nerves, potentially due to modifications of the membrane potential threshold upon the affection/disease [Amir, R. et al., (1999) Membrane Potential Oscillations in Dorsal Root Ganglion Neurons: Role in Normal Electrogenesis and Neuropathic Pain, The Journal of Neuroscience, 19 (19):8589-8596]. Increased Na⁺ conductance, caused by nerve injury-induced changes in the vectorial transport of Na⁺ channels and/or Na⁺ channel upregulation, is implicated as a factor in the generation of neuropathic (ectopic) afferent discharge in animals and humans.

Thus, we see that NP may result from disorders of the peripheral nervous system or the central nervous system (brain and spinal cord). Thus, NP may be divided into peripheral NP, central NP, or mixed (peripheral and central) NP.

Trigeminal Neuralgia, painful radiculopathy, postherpetic neuralgia, painful polyneuropathy, including Chemotherapy Induced Peripheral Neuropathy (CIPN, also called Chemotherapy-Induced Neuropathic Pain or CINP) and diabetic polyneuropathy, and peripheral nerve injury pain are classed as peripheral NP. By contrast, central NP includes painful conditions such as spinal cord injury (SCI) neuropathic pain, central pain in multiple sclerosis and central post-stroke pain (see FIG. 4 of Finnerup, N. B, et al. (2021) Neuropathic Pain: From Mechanisms To Treatment, Physiol Rev 101: 259-301).

Currently used clinical solutions to treat NP may be systemic treatment or local treatment. For example, gabapentinoid drugs, tricyclic antidepressants (TCAs) and selective serotonin-norepinephrine reuptake inhibitors (SNRIs), all of which are systemic treatments, are usually considered as first line treatments.

Gabapentinoid drugs, Gabapentin and Pregabalin, bind to Ca²⁺ voltage-dependent channels, thereby reducing Ca²⁺ influx to the cells, and are used in the treatment of diabetic pain, post-herpetic neuralgia, SCI, and phantom limb syndrome. However, both Gabapentin and Pregabalin are associated with side effects that include lethargy, vertigo, peripheral nerve swelling and blurred vision.

TCAs are used to treat nerve injury pain, post-herpetic neuralgia, central postpartum pain, and in the treatment of pain following SCI. However, TCAs and SNRIs are contraindicated for patients having cardiac problems. They are also associated with several side effects including nausea, and lethargy. Opioids, including Tramadol and Tapenadol, have been proposed as the second-line treatment of NP, but are associated with side effects such as nausea/vomiting, constipation, and lethargy. Drug habituation/addiction and drug abuse also constitute a major issue with opioid treatment.

Topical treatments, Lidocaine and Capsaicin, are used in the local treatment of neuropathic pain and are generally applied as patches. Capsaicin (what makes chilli peppers hot) is a vanilloid receptor 1 agonist. The application of the capsaicin containing patch is carried out by a physician and may be painful, a burning sensation on the skin frequently being reported [https://www.vidal.fr/medicaments/qutenza-179-mg-patch-cutane-gel-nettoyant-93564.html]. The single application is designed to produce pain relief for up to approximately three months.

Third-line treatments for NP include systemic treatments—Tramadol, strong opioids (Morphine and Oxycodone) and locally applied botulinum toxin-A (BTX-A). Intradermal injection of BTX-A is currently used in the treatment of chronic focal painful neuropathies. Botulinum toxin A can be administered via multiple intradermal injections, the affected painful area being divided into a chessboard of multiple sites, with typically 5 U of BTX-A injected per site [Park, J H., et al. (2017) Botulinum toxin for the treatment of neuropathic pain, Toxins, 9, 260]. However, botulinum toxin-A causes muscle paralysis and therefore may impact quality of life [Mittal, S O et al. (2016) Botulinum Toxin Treatment of Neuropathic Pain, Seminars in Neurology. 36 (1): 73-83].

Overall, despite treatments listed above, neuropathic pain is difficult to treat with only about 40-60% of patients achieving partial relief [Dworkin, R H, et al. (2007) Pharmacologic management of neuropathic pain: evidence-based recommendations, Pain, 132 (3): 237-51].

Several physics-based approaches have been evaluated in the clinic for patients with refractory neuropathic pain. These approaches include non-invasive transcranial brain stimulation techniques, like repetitive transcranial magnetic stimulation (rTMS), wherein electrical currents are produced in the cortex through a transient magnetic field. In transcranial direct current stimulation (tDCS) a low voltage current (1-2 mA) is applied transcranially. TENS and PENS are peripheral electrical stimulation methods that have some use in the treatment of NP. Repetitive spinal magnetic stimulation (SMS) and frequency-modulated electromagnetic stimulation (FREMS) are two magnetic based techniques that have been tested in the treatment of NP. The general disadvantage of the latter external stimulation techniques is that their efficacy is limited in time (days or weeks) and restimulation is usually required for a sustained effect. Dorsal column stimulation (spinal cord stimulation, SCS) is an invasive technique used to suppress the central neuronal hyperexcitability. The spinal cord dorsal column is stimulated electrically by 50 Hz pulses from an implanted generator in the spinal column. Patients undergoing this treatment are typically those who cannot obtain pain relief with other non-invasive methods. Zeng, H. et al. recently reviewed the non-invasive neuro modulation (NINM) effects on peripheral diabetic neuropathy (PDN, or diabetes-induced neuropathic pain, DNP) and found that NINMs effects were higher with intensive protocols and in populations with resistant symptoms or intolerance to analgesic medication [Zeng, H. et al. (2020) Non-invasive neuromodulation effects on painful diabetic peripheral neuropathy: a systematic review and meta-analysis. Scientific Reports 10.]

In view of the foregoing, there is a need for new treatments for patients suffering from NP and researchers continue to explore new treatment methods. For example, patent application US20210402004A1 describes the use of targeted cerium-containing nanoparticles for the treatment of NP via the protection of microglia from oxidative activation. The authors indicate that the conversion of Ce³⁺/Ce⁴⁺ oxidation states allows the particles to scavenge reactive oxygen species (ROS) and down-regulate the hyperactivated microglia. However, a disadvantage of the latter system may be the well-known cellular toxicity associated with the ROS activity of cerium oxide nanoparticles [Kumari, M, et al. (2014) Toxicity study of cerium oxide nanoparticles in human neuroblastoma cells. Int J Toxicol. 33(2):86-97.].

Thus, there is a continued need for new NP treatments having reduced side effects compared to those associated with the currently available treatments, as described above. Furthermore, there is a need for treatments with relatively long-lasting efficacy, so that the patient can experience reduced pain for as long as possible, for example, for longer than three months (which is the current average time during which local treatments are efficacious).

In particular, there is a need for treatment of peripheral neuropathic pain conditions. There is a need for treatments for patients suffering from peripheral diabetic neuropathy (PDN or DNP). There is a need for treatments for patients suffering from post-herpetic neuralgia. There is a need for treatments for patients suffering from Chemotherapy Induced NP, or CIPN or CINP. There is a need for treatments for patients suffering from peripheral nerve injury NP that is post-traumatic and/or post-surgery and/or neuroma-induced NP.

There is a need for treatment of focal neuropathic pain. There is a need for treatment of refractory and/or chronic neuropathic pain.

There is a need for treatments for patients suffering from peripheral neuropathic pain. There is a need for treatments for patients suffering from NP resulting from autoimmune diseases affecting the peripheral nervous system (PNS), like Guillain-Barre syndrome. There is a need for treatments for patients suffering from ectopic afferent discharges associated NP. Generally, there is a need for treatments for the aforementioned patients so that their perceived pain is decreased.

The present invention advantageously offers a solution to treat patients suffering from NP, in particular, peripheral NP, using, non-toxic (chemically inert) and non-biodegradable nanoparticles unlike those described in the prior art. The treatment is administered locally with minimal discomfort, is minimally invasive, has a long-lasting effect without any of the side effects associated with currently available systemic treatments. Thus, the invention provides an advantageous solution to each of the herein above-described needs.

### SUMMARY OF THE INVENTION

In one aspect, the invention concerns nanoparticles or aggregates thereof for use in treating neuropathic pain (“NP”).

In an aspect, the invention concerns a method of treatment of a patient suffering from neuropathic pain.

In one aspect of the invention, the nanoparticles may be used to treat patients suffering from trigeminal neuralgia, neuropathic pain following peripheral nerve injury including post-traumatic neuropathic pain, post-surgical neuropathic pain or neuroma-induced neuropathic pain, painful polyneuropathy associated with diabetes, human immunodeficiency virus (HIV), Fabry disease, sodium channel gene mutation(s), autoimmune diseases like Guillain-Barré syndrome, vasculitis, chronic inflammatory demyelinating polyneuropathy, amyloidosis, nonfreezing cold injury, paraneoplastic syndrome or leprosy, chemotherapy-induced peripheral neuropathy (CIPN), post herpetic neuralgia, or painful radiculopathy.

In a particular aspect, the nanoparticles may be used to treat patients suffering from peripheral nerve injury including post-traumatic neuropathic pain, post-surgical neuropathic pain or neuroma-induced neuropathic pain, post herpetic neuralgia, painful polyneuropathy associated with diabetes, CIPN, or HIV.

In a preferred aspect of the invention, the nanoparticles may be used to treat patients suffering from chemotherapy-induced peripheral neuropathy (CIPN).

In another preferred aspect of the invention, the nanoparticles may be used to treat patients suffering from diabetes-induced polyneuropathy also herein identified as “painful polyneuropathy associated with diabetes” or “peripheral diabetic neuropathy”.

In another aspect of the invention, the nanoparticles may be used to treat patients suffering from neuropathic pain following peripheral nerve injury including post-traumatic neuropathic pain, post-surgical neuropathic pain or neuroma-induced neuropathic pain.

In another aspect of the invention, the nanoparticles may be used to treat patients suffering from ectopic afferent discharges associated neuropathic pain.

There is a need for treatments for patients suffering from peripheral neuropathic pain.

In another aspect of the invention, the nanoparticles may be used to treat patients suffering from focal neuropathic pain.

In another aspect of the invention, the nanoparticles may be used to treat patients suffering from refractory and/or chronic neuropathic pain.

In one aspect of the description, the nanoparticle or nanoparticles aggregate material may be selected from an insulator material with a dielectric constant εᵢⱼₖ equal to or below 100 selected from a metal oxide and a mixed metal oxide, wherein the metal is chosen from Zr, Hf and/or Re, for example HfO₂ and/or ZrO₂. In a particular aspect, the material of the nanoparticle or nanoparticles aggregate is a metal oxide or a mixed metal oxide, wherein the metal is Zr and/or Re.

In one aspect of the invention, the nanoparticle or nanoparticles aggregate material may be a metal conductor selected from Ir, Pd, Pt, Au and any mixture thereof.

In another aspect, the nanoparticle or nanoparticles aggregate material may be an organic material having contiguous sp² hybridized carbon centers in its structure (i.e., carbon double bond or aromatic cycles comprising heteroatoms, typically N or S, within the aromatic cycle or outside the aromatic cycle). Preferred organic materials are selected from polyaniline, polypyrrole, polyacetylene, polythiophene, polycarbazole, polystyrene and/or polypyrene. More preferred organic material is poly(3,4-ethylenedioxythiophene) (PEDOT), poly(styrene sulfonate) (PSS) or a mixture thereof.

In one aspect of the invention, when the nanoparticle material is a metallic conductor, the nanoparticle or nanoparticle aggregate has a median population core size of at least 45 nm.

In one aspect of the invention, the core of the nanoparticle or nanoparticles' aggregate is coated with a biocompatible coating providing a neutral or a negative surface charge when measured in a solution of water having an electrolyte concentration between 0.001 and 0.2 M, a concentration of the nanoparticles' or nanoparticles' aggregates' material between 0.01 and 10 g/L and a pH between 6 and 8.

In another aspect, the nanoparticles or aggregate thereof may be administered by intradermal and/or intraepidermal injection. The nanoparticles or aggregate(s) thereof may be administered using a dissolvable microneedle patch.

In another aspect of the invention, the dose of nanoparticles or aggregates thereof administered is preferably at least 60 μg/cm² of skin surface to be injected.

In another aspect of the invention, when the patient suffers from painful polyneuropathy associated with diabetes, the dose of nanoparticles or aggregate thereof administered is preferably at least 60 μg/cm² of skin surface to be injected.

In one aspect of the description, when the patient suffers from CIPN and when the material of the nanoparticles or aggregate thereof is selected from a metal oxide or mixed metal oxide, for example HfO₂ and/or ZrO₂, the dose of nanoparticles or aggregate thereof administered is preferably at least 250 μg/cm², more preferably, at least 390 μg/cm² of skin surface to be injected.

In another aspect, when the patient suffers from CIPN and when the nanoparticles or aggregate thereof is selected from a metal conductor selected from Ir, Pd, Pt, Au and any mixture thereof, the dose of nanoparticles or aggregate thereof administered is preferably at least 70 μg/cm², for example 78 μg/cm², of skin surface to be injected.

### FIGURES

FIG. 1: CIPN model (Example 5): Mechanical allodynia. PWT (g) using von Frey filaments in test groups at different time points. *, **, ***, and **** indicate statistically significant difference respectively with P<0.05, P<0.01, P<0.001, and P<0.0001, when each group is compared to its own value at the ‘NP’ timepoint (neuropathic baseline) using Dunnett's Multiple Comparison Test following a mixed-effects analysis. Values are mean±SEM.

FIG. 2: CIPN model (Example 5): Pictures of a rat hind paw after intraplantar injection of Nanoparticles from Example 1 (gold nanoparticles) showing the diffusion of nanoparticles into the skin, with no evolution between D7 and D26

FIG. 3: CIPN model (Example 6): Mechanical Allodynia. Paw withdrawal threshold (g) using von Frey filaments in the Vehicle Group (naïve animals) at different time points. Values are mean±SEM.

FIG. 4: CIPN model (Example 6): Mechanical Allodynia. Paw withdrawal threshold (g) using von Frey filaments in the Nanoparticles 61 g/L/20 μL Group at different time points. Left paw (white bars): injection of 20 μL of Nanoparticles 61 g/L; right paw (grey bars): injection of 20 μL of Vehicle (saline). Values are mean±SEM.

FIG. 5: CIPN model (Example 6): Mechanical Allodynia. Paw withdrawal threshold (g) using von Frey filaments in the Nanoparticles 18 g/L/20 μL Group at different time points. Left paw (white bars): injection of 20 μL of Nanoparticles 18 g/L; right paw (grey bars): injection of 20 μL of Vehicle (saline). Values are mean±SEM.

FIG. 6: CIPN model (Example 6): Mechanical Allodynia. Paw withdrawal threshold (g) using von Frey filaments in the Nanoparticles 18 g/L/10 μL Group at different time points. Left paw (white bars): injection of 10 μL of Nanoparticles 18 g/L; right paw (grey bars): injection of 10 μL of Vehicle (saline). Values are mean±SEM.

FIG. 7: CIPN model (Example 6): Mechanical Allodynia. Paw withdrawal threshold (g) using von Frey filaments in the Pregabalin Group at different time points. Systemic administration of Pregabalin (per os); no difference between left and right paw. Values are mean±SEM.

FIG. 8: STZ-induced diabetic NP study (Example 7): Mechanical Allodynia. Paw withdrawal threshold (g) using von Frey filaments at different time points. Values are mean±SEM.*, **, ***, and **** respectively indicate statistically significant difference with p<0.05, p<0.01, p<0.001, and p<0.0001 when compared to the ‘STZ/Vehicle’ Group at each timepoint. Values are mean±SEM.

### DETAILED DESCRIPTION

### Definitions

The terms “treatment” or “therapy” refer to both therapeutic and prophylactic or preventive treatment or measures that can significantly slow disease progression, or treat symptoms (for example reduce pain).

Such a treatment or therapy is intended for a subject in need thereof, typically a human being (also herein identified as a human patient, or a patient).

“Focal neuropathic pain” means neuropathic pain that is limited in surface area to less than 15 cm².

“Refractory neuropathic pain” means neuropathic pain that is long-term and that is not relieved by standard pharmacological intervention, like standard anti-depressant drugs, anti-convulsant drugs, analgesic drugs, etc. Experts recognize that the definition “refractory neuropathic pain” means NP with a minimum duration one year; at least four drugs of known effectiveness tried for three months or the maximum tolerated time [Smith B. H., et al. (2012) Towards a definition of refractory neuropathic pain for epidemiological research. An international Delphi survey of experts, BMC Neurology, 12:29].

“Chronic neuropathic pain” means regularly occurring neuropathic pain over a period of several months [J. Smith et al. (2018) J. Neurol. 265, pp231-238].

“At least partial thermal sensation” means preservation of the afferent fibers that allow the patient feel “evoked” pain, more specifically, cold or warm allodynia/hyperalgesia.

Thermal sensation, more specifically cold and/or warm allodynia and/or hyperalgesia may be measured according to methods known to the skilled person and include protocols for Quantitative Sensory Testing, like the DFNS (German Research Network on Neuropathic Pain) protocol [Vollert, J., et al. (2015) Quantitative Sensory Testing using DFNS protocol in Europe: an evaluation of heterogeneity across multiple centers in patients with peripheral neuropathic pain and healthy subjects, Pain, 157(3)] and the NPSI (Neuropathic Pain Symptom Inventory) protocol [Bouhassira, D., et al. (2004) Development and validation of the Neuropathic Pain Symptom Inventory, Pain, 108:248-257].

“Thermal sensation”, or “at least partial thermal sensation”, means that the patient still has (partially) preserved (epidermal) afferent fibers.

“Levels of neuropathic pain” means levels of pain that are measurable using standard questionnaires known to the skilled person, including the Numerical Rating Scale for pain (NRS), the Leeds Assessment of Neuropathic Symptoms and Signs (LANSS), the Neuropathic Pain Questionnaire, the DN4, the pain DETECT questionnaire, the ID pain, the Neuropathic Pain Scale (NPS), the DFNS protocol and the Neuropathic Pain Symptom Inventory (NPSI) [HaanpÅÅ, M., et al. (2011) NeuPSIG guidelines on neuropathic pain assessment, Pain 152 14-27]. Preferably, the NRS for pain and/or the DFNS protocol and/or the NPSI is/are used.

Nanoparticles and/or Aggregates of Nanoparticles

### Size

In the context of the invention, the term “nanoparticle” refers to a product, in particular, a synthetic product, with a size in the nanometer range, typically between about 1 nm and about 1000 nm, preferably between about 1 nm and about 500 nm, even more preferably, between about 1 nm and about 100 nm.

According to one aspect of the invention, the median largest size of the core of the nanoparticle or nanoparticles' aggregate of the population is between about 10 nm and about 200 nm. The term “aggregate of nanoparticles” refers to an assemblage of nanoparticles.

The size of the nanoparticle and/or aggregates of nanoparticle can typically be measured by Electron Microscopy (EM) technics, such as transmission electron microscopy (TEM) or cryo-TEM, as well known by the skilled person. The size of at least 100 nanoparticles and/or aggregates of nanoparticles is typically measured and the median size of the population of nanoparticles and/or aggregates of nanoparticles is reported as the size of the nanoparticle and/or aggregate of nanoparticles.

### Shape

As the shape of the nanoparticles and/or aggregates of nanoparticles can influence their “biocompatibility”, nanoparticles and/or aggregates of nanoparticles having a quite homogeneous shape are preferred. For pharmacokinetic reasons, nanoparticles and/or aggregates of nanoparticles being essentially spherical, round or ovoid in shape are thus preferred. Such a shape also favors the nanoparticle and/or aggregates of nanoparticles interaction with, or uptake by, cells.

### Composition/Structure

Nanoparticle Prepared from an Insulator Material Having a Low Relative Dielectric Constant (Relative Permittivity), i.e., Equal to or Below 100

The nanoparticles prepared from (e.g. comprising), or consisting of, an insulator material having a low relative dielectric constant are typically prepared with a material having a band gap Eg equal to or above 3.0 eV typically when measured at room temperature (about 25° C.), and a relative dielectric constant εᵢⱼₖ equal to or below 100, preferably below 50 or below 20, which is typically measured between 20° C. and 30° C. and between 10² Hz up to the infrared frequency (see for instance table 12-45 “Permittivity (dielectric constant) of inorganic solid”; Handbook of chemistry and physics; David R. Lide; 88^{th} Edition; Compilation of the static dielectric constant of inorganic solid. K. F. Young and H. P. R. Frederikse. J. Phys. Chem. Ref. Data, Vol. 2, No. 2, 1973).

Such nanoparticles are typically prepared with a dielectric material which is selected from a metal oxide, a mixed metal oxide, the metallic element of which is from period 3, 5 or 6 of the Mendeleev's periodic table or a lanthanide, and a carbon material. The dielectric material is preferably selected from La₂O₃, SnO₂, Ta₂O₅, ReO₂, ZrO₂, HfO₂ and carbon diamond. More preferably, the dielectric material is a metal oxide selected from ZrO₂, HfO₂ and any mixture thereof. Particularly preferred is a dielectric material selected from ZrO₂ and HfO₂.

In a particular and preferred aspect, the metal oxide is not CeO₂ (cerium oxide). Cerium oxide is also known as “ceria”. As mentioned in the preamble, cerium oxide is known to have cytotoxic effect. Indeed Pulido-Reyes et al. (2015) proved that the main driver for the toxicity for ceria nanoparticles is the percentage of surface content of Ce³⁺ sites, with a higher Ce³⁺/Ce⁴⁺ ratio leading to higher toxicity [“Untangling the biological effects of cerium oxide nanoparticles: the role of surface valence states,” Scientific reports DOi: 10.1038/srep15613]. It is also known from Soh et al. 2017 [Angew. Chem. Int. Ed. 10.1002/anie.201704904]. Therefore, incorporating Zr⁴⁺ into cerium oxide lattice, which favors the conversion of Ce⁴⁺ to Ce³⁺ at the surface of the ceria zirconium surface, leads to greater nanoparticle toxicity. Thus, one expects cerium/zirconium oxide nanoparticles to be more toxic than cerium oxide nanoparticles and zirconia nanoparticles because of redox reactions occurring at their surface, compared to known surface inertness of zirconium oxide nano-biomaterial [Shtansky et al., (2015): Multifunctional bioactive nanostructured films in “Hydroxyapatite (HAp) for Biomedical Applications” Elsevier, pp 162].

In another particular and preferred aspect, the metal oxide or the mixed metal oxide does not contain cerium (Ce).

In a particular aspect, when the material is selected from ReO₂, ZrO₂ and HfO₂, preferably from ZrO₂ and HfO₂, (for example is ReO₂ or ZrO₂), the median largest size of the core of the nanoparticle or nanoparticles' aggregate of the population is of at least 10 nm and below 500 nm, preferably between about 10 nm and about 200 nm, even more preferably, between about 20 and 100 nm.

Nanoparticle Prepared from a Conductor Material

The nanoparticle prepared from a conductor material is an organic nanoparticle or an inorganic nanoparticle.

Inorganic nanoparticle prepared from a conductor material is typically prepared with a metallic element having a standard reduction potential E° value equal to or above about 0.01, typically when measured at 25° C. and at a pressure of 1 atm in respect to the standard hydrogen electrode (see Table 2 “reduction reactions having E° values more positive than that of the standard hydrogen electrode”, 8-25, Handbook of chemistry and physics; David R. Lide; 88^{th} Edition), more preferably equal to or above about 0.1, 0.2, 0.3, 0.4, or 0.5. Typical metallic elements used to prepare the nanoparticles may be selected from Pd, Ir, Pt, Au and any mixture thereof. Preferably, the metallic element usable as conductor material to prepare the nanoparticles is selected from Ir, Pd, Pt, Au, and any mixture thereof, even more preferably is selected from Au, Pt, Pd and any mixture thereof. Particularly preferred materials are Au and Pt.

Typically, gold nanoparticles have shown catalytic activity when their size was decreased to few nm (Auffan, M., et al. (2009) Towards a definition of inorganic nanoparticles from an environmental, health and safety perspective, Nature Nanotechnology, 4(10), 634-641). In order to reduce the surface/volume ratio and thus minimize the contribution of the gold nanoparticle's surface to the catalytic activity, a median largest size of the core of the nanoparticle or of the nanoparticles' aggregate of the population of at least 30 nm, typically of at least 40 nm or at least 45 nm is preferred.

In a particular aspect, when the material is a metallic material, typically a metal having a standard reduction potential E° above 0.2, in particular, any one of Ir, Au, Pt, Pd and any mixture thereof, the median largest size of the core of the nanoparticle or nanoparticles' aggregate of the population is of at least 30 nm, or of at least 40 nm and preferably, below 500 nm as described herein above. For example, the median largest size of the core of the nanoparticle or nanoparticles' aggregate of the population may be between about 40 and about 200 nm, preferably between about 45 and about 100 nm.

Organic nanoparticles prepared from a conductor material is typically prepared with an organic material having contiguous sp² hybridized carbon centers in its structure (i.e., carbon double bond or aromatic cycles comprising heteroatoms, typically N or S, within the aromatic cycle or outside the aromatic cycle). Preferred organic materials are selected from polyaniline, polypyrrole, polyacetylene, polythiophene, polycarbazole, polypyrene, poly(3,4-ethylenedioxythiophene) and/or poly(3,4-ethylenedioxythiophene) polystyrene sulfonate. More preferably, organic materials are poly(3,4-ethylenedioxythiophene) or poly(3,4-ethylenedioxythiophene) polystyrene sulfonate. In another preferred aspect, organic materials are poly(3,4-ethylenedioxythiophene) (PEDOT) and/or poly(styrene sulfonate) (PSS).

### The Nanoparticles' or Aggregates of Nanoparticles' Biocompatible Coating

In a preferred embodiment, the core of the nanoparticle or nanoparticles' aggregate used in the context of the present invention to prepare a composition of interest can be coated with a biocompatible material bringing a negative charge on the nanoparticle's or nanoparticles' aggregate's surface. An agent forming a negative charge on the nanoparticle's or nanoparticles' aggregate's surface can be for example a phosphate (for example a polyphosphate, a metaphosphate, a pyrophosphate, etc.), a carboxylate (for example citrate or dicarboxylic acid, in particular succinic acid) or a sulphate.

In a preferred embodiment, the core of the nanoparticle or aggregate of nanoparticles presents a hydrophilic neutral surface charge or is coated with a biocompatible material (i.e. a coating agent) selected from a hydrophilic agent conferring a neutral surface charge to the nanoparticle.

A hydrophilic agent conferring neutral surface charge to the core of the nanoparticle or nanoparticles' aggregate may be an agent displaying a functional group selected from an alcohol (R—OH), an aldehyde (R—COH), a ketone (R—CO—R), an ester (R—COOR), an acid (R—COOH), a thiol (R—SH), a saccharide (glucose, fructose, ribose for instance), an anhydride (RCOOOC—R), and a pyrrole. The hydrophilic agent conferring a neutral surface charge to the core of the nanoparticle or nanoparticles' aggregate can be a monomer, a dimer, an oligomer, a polymer or a copolymer. When the agent is an oligomer, it may be an oligosaccharide such as a cyclodextrin. When the agent is a polymer, it may be a polyester (such as a poly(lactic acid) or a polyhydroxyalkanoic acid), a polyether, a polyethylene oxide, a polyethylene glycol, a polyvinylalcohol, a polycaprolactone, a polyvinylpyrrolidone, a polysaccharide such as a cellulose, a polypyrrole, etc.

In addition, a hydrophilic agent conferring neutral surface charge to the core of the nanoparticle or nanoparticles' aggregate may be an agent displaying specific groups (X—) able to interact with the surface of the nanoparticle or aggregate of nanoparticles. X is typically selected from a thiol, a silane, a carboxylic and a phosphate group.

When the core of the nanoparticle or aggregate of nanoparticles is a conductor and a metallic nanoparticle, X is preferably a thiol, a thioether, a thioester, a dithiolane or a carboxylic group. Preferably, the hydrophilic neutral coating agent is selected from a thioglucose, a 2-mercaptoethanol, a 1-thioglycerol, a thiodiglycol, a hydroxybutyric acid and a mercaptopolyethylene glycol. Preferably, the hydrophilic agent conferring a negative surface charge at physiological pH is a dimercaptosuccinic acid.

When the core of the nanoparticle or aggregate of nanoparticles is an insulator, a metal oxide or a mixed-metal oxide nanoparticle, X is preferably a silane or a phosphate group. Preferably, the hydrophilic neutral coating agent is a silane-polyethylene glycol. Preferably, the hydrophilic agent conferring a negative surface charge at physiological pH is a polyphosphate molecule, more preferably hexametaphosphate.

According to a preferred aspect herein described, the core of the nanoparticle or nanoparticles' aggregate consists of HfO₂. Preferably, the core is coated with a biocompatible coating bringing a negative surface charge, for example with a coating displaying a phosphate group. In one aspect herein described a core HfO₂ is coated with a hexametaphosphate biocompatible coating.

According to one embodiment of the invention, the core is coated with a biocompatible hydrophilic neutral coating agent selected from a hydroxymethyltriethoxysilane, a fructose 6-phosphate or a glucose 6-phosphate compound.

A hydrophilic agent conferring neutral surface charge to the core of the nanoparticle or nanoparticles' aggregate may be a zwitterionic compound such as an amino acid, a peptide, a polypeptide, a vitamin or a phospholipid.

The surface charge of a nanoparticle or nanoparticles' aggregate is typically determined, as well known by the skilled person, by zeta potential measurements, typically in (a solution of) water having a concentration of nanoparticles' or nanoparticles' aggregates' material between 0.01 and 10 g/L, a pH between 6 and 8, and typically a concentration of electrolytes (in water) between 0.001 and 0.2 M, for example 0.01 M or 0.15 M. Under the herein above defined conditions, the surface charge of the nanoparticle or aggregate of nanoparticles is typically comprised between −80 mV and +15 mV, between −60 mV and +10 mV, or between −45 mV and +10 mV. When neutral, the surface charge of the nanoparticles or aggregate of nanoparticles is typically comprised between −10 mV, −9 mV, −8 mV, −7 mV, −6 mV, −5 mV, −4 mV, −3 mV, −2 mV, or −1 mV and 1 mV, 2 mV, 3 mV, 4 mV, 5 mV, 6 mV, 7 mV, 8 mV, 9 mV or 10 mV. When negative, the surface charge of the nanoparticles or aggregate of nanoparticles is typically below −11 mV, −12 mV, −13 mV, −14 mV −15 mV, −16 mV, −17 mV, −18 mV, −19 mV, −20 mV, −21 mV, −22 mV, −23 mV, −24 mV, −25 mV, −26 mV, −27 mV, −28 mV, −29 mV, −30 mV, −31 mV, −32 mV, −33 mV, −34 mV, −35 mV, −40 mV, or −45 mV.

A full biocompatible coating of the nanoparticle or aggregate may be advantageous in the context of the present invention in order to avoid any electrical charge on the nanoparticle's surface, when the nanoparticle presents a hydrophilic neutral surface charge. The “full coating” implies the presence of a very high density/compactness of biocompatible molecules able to create at least half a monolayer, or preferably, at least a complete monolayer on the surface of the particle.

The biocompatible coating allows in particular the nanoparticle's stability in a fluid, such as a physiological fluid (blood, plasma, serum, etc.) or any isotonic media or physiologic medium required for a pharmaceutical administration.

Stability may be confirmed by dry extract quantification using a drying oven and measured on a nanoparticle suspension prior and after filtration, typically on a 0.45 μm filter.

Advantageously, the coating preserves the integrity of the particle in vivo, ensures or improves the biocompatibility thereof, and facilitates an optional functionalization thereof (for example with spacer molecules, biocompatible polymers, targeting agents, proteins, etc.).

The biocompatible nanoparticle or aggregate of nanoparticles of the invention should neither dissolve thereby releasing potentially toxic species following in vivo administration (i.e. at physiological pH) nor present redox behavior, typically for said nanoparticle or aggregate of nanoparticles to be considered biocompatible, i.e. to be safely used in a subject, in particular in a mammal, preferably in a human being.

Another particular object herein described relates to a composition, in particular a pharmaceutical composition, comprising nanoparticles and/or nanoparticles' aggregates such as described hereinabove, preferably together with a pharmaceutically acceptable carrier or vehicle.

In a particular aspect, the composition can comprise the nanoparticles or nanoparticles' aggregates of the invention together with a therapeutic agent, for example, for local administration, like an anti-inflammatory drug.

The composition can be in the form of a solid, liquid (particles in suspension), aerosol, gel, paste, and the like. Preferred compositions are in a liquid or a gel form. Particularly preferred compositions are in liquid form.

The pharmaceutically acceptable support or carrier which is employed can be any classical support for the skilled person, such as for example a saline, isotonic, sterile, buffered solution, a non-aqueous vehicle solution and the like.

The composition can also comprise stabilizers, surfactants, polymers and the like.

It can be formulated for example as ampoule, or liquid for injection by using techniques of pharmaceutical formulation known by the skilled person.

### Subjects to be Treated:

The nanoparticles are administered for the treatment of at least one peripheral neuropathic pain condition.

According to one embodiment of the invention, the subject suffers from peripheral diabetic neuropathy (PDN).

According to one embodiment of the invention, the subject suffers from post-herpetic neuralgia.

According to one embodiment of the invention, the subject suffers from CIPN.

According to one embodiment of the invention, the subject suffers from painful radiculopathy.

According to one embodiment of the invention, the subject suffers from peripheral nerve injury including post-traumatic neuropathic pain, post-surgical neuropathic pain or neuroma-induced neuropathic pain.

According to one embodiment of the invention, the patient suffers from (peripheral) neuropathic pain due to an autoimmune disease like Guillain-Barré syndrome, human immunodeficiency virus (HIV), Fabry disease, sodium channel gene mutation(s), vasculitis, chronic inflammatory demyelinating polyneuropathy, amyloidosis, nonfreezing cold injury, paraneoplastic syndrome or leprosy.

According to one embodiment of the invention the subject suffers from a focal (peripheral) neuropathic pain.

According to one embodiment of the invention the subject suffers from a refractory and/or chronic (peripheral) neuropathic pain.

According to a preferred embodiment of the invention, the subject suffers from peripheral NP manifesting as ectopic afferent discharges.

The subject suffering from NP may preferably have at least partial thermal sensation preserved (tested via cold or warm allodynia/hyperalgesia tests known from the skilled person).

Without being bound by any theory, the Inventors believe that the preservation of at least some thermal sensation, meaning preservation of at least some of the (epidermal) afferent fibers, may be necessary in the subjects for the sensation of pain relief to be felt by the treated subject.

### Mode of Administration:

The affected painful area may be delineated by the skilled person (i.e. a physician or a nurse), drawn on the skin with a pen and measured. This delineation may help to determine the total dose of nanoparticles to be administered in the painful area. The as-delineated painful area should preferably be inferior to 15 cm².

The nanoparticles or nanoparticles' aggregates of the invention can be administered to the subject using intradermal and/or intraepidermal injection. Intradermal injection can be performed with syringes usually used for intradermal injection (insulin or tuberculin syringe, for example), with a 25 to 30 needle gauge, at the angle usually used for intradermal injection (5- to 15-degree angle from the site, for example). Intradermal injection can also be performed according to the Mantoux test. In another aspect, intradermal and/or intraepidermal injection can be performed using hollow, solid, or dissolvable microneedles. Such microneedles can be in the form of a patch [Jung, J. H., et al. (2021) Microneedle for transdermal drug delivery: current trends and fabrication, Journal of Pharmaceutical Investigation, 51, 503-517]. The nanoparticles or nanoparticles' aggregates of the invention can be administered via multiple intradermal and/or intraepidermal injections, the affected painful area being divided into a chessboard of multiple sites, with a dose per unit site, the way it can be usually performed with Botulinum toxin A to treat focal neuropathic pain.

The nanoparticles or nanoparticles' aggregates may be administered as often as necessary according to the patient's pain. The nanoparticle administration may be necessary once every month, or every two months or every three months.

Preferably, the nanoparticle administration is necessary only once every three months. More preferably, the nanoparticle administration is necessary only once every four months. Even more preferably, the nanoparticle administration is necessary only once every five or six months. Even more preferably, the nanoparticle administration is necessary only once every seven or eight months. Most preferably, the nanoparticles are administered only once and provide pain relief for more than a year. Even most preferably, the nanoparticles are administered only once and provide pain relief for the rest of patient's life.

The nanoparticles and/or nanoparticles' aggregates once administered typically interact with the subject's neurons/nerves. In a preferred aspect, this interaction is a prolonged interaction, i.e., an interaction of several hours, days, weeks or months. In a particular aspect, the nanoparticles or nanoparticles' aggregates remain in the subject.

The herein described nanoparticles or nanoparticles' aggregates and compositions comprising such nanoparticles or nanoparticles' aggregates are for use in a subject, typically for use in a human patient.

Typical number of nanoparticles or aggregates of nanoparticles to be administered in the dermis and/or epidermis of the subject is between 10⁵ and 10¹⁷, between 10⁵ and 10¹⁶ or between 10⁵ and 10¹⁵, preferably between 10⁷ and 10¹⁴, more preferably between 10⁹ and 10¹³ per cm². Most preferably, typical number of nanoparticles or aggregates of nanoparticles to be administered in the dermis and/or epidermis of the subject is between 10¹⁰ and 10¹² per cm².

According to one embodiment of the invention, when the patient suffers from painful polyneuropathy associated with diabetes, the dose of nanoparticles or aggregate thereof administered is preferably at least 60 μg/cm² of skin surface to be injected.

In one aspect, when the patient suffers from polyneuropathy associated with diabetes and when the material of the nanoparticles or aggregate thereof is a metal oxide or a mixed metal oxide, for example HfO₂ and/or ZrO₂, the dose of nanoparticles or aggregate thereof administered is preferably, at least 60 μg/cm² of skin surface to be injected.

In one aspect, when the patient suffers from CIPN and when the material of the nanoparticles or aggregate thereof is a metal oxide or a mixed metal oxide, for example HfO₂ and/or ZrO₂, the dose of nanoparticles or aggregate thereof administered is preferably, at least 250 μg/cm², more preferably, at least 390 μg/cm² of skin surface to be injected.

According to one embodiment, when the patient suffers from CIPN, and when the material of the nanoparticles or aggregate thereof is a metal conductor selected from Ir, Pd, Pt, Au and any mixture thereof, the dose of nanoparticles or aggregate thereof administered is preferably at least 70 μg/cm², for example 78 μg/cm², of skin surface to be injected.

### Targeting

A particular nanoparticle and/or aggregate of nanoparticles as herein described further comprises a targeting agent allowing its interaction with a recognition element present on a target cell, typically on a neuron/nerve cell. Such a targeting agent typically acts once the nanoparticles and/or aggregates of nanoparticles are accumulated on the target site, typically in the skin, even more typically on neurons/nerves. The targeting agent can be any biological or chemical structure displaying affinity for molecules present in the human or animal body. For instance, it can be a peptide, oligopeptide or polypeptide, a protein, a nucleic acid (DNA, RNA, SiRNA, tRNA, miRNA, etc.), an antibody, a hormone, a vitamin, an enzyme, the ligand of a molecule/receptor expressed by a neuron/nerve cell or by a pathological cell, in particular the ligand of an intraepidermal nerve fiber receptor, the ligand of an intradermal nerve fiber receptor, the ligand of an axon receptor, of a soma receptor, of a Schwann cell receptor, the ligand of myelin, the ligand of an axonal transport vesicle/endosome receptor, the ligand of a signaling endosome receptor, the ligand of neurotrophins, of semaphorins, of nerve growth factor(s), of growth cone receptor(s) or of netrin receptor complexes, the ligand of a receptor protein tyrosine phosphatase, the ligand of dynein or kinesin, or of a cytokine receptor. Said targeting agent can be selected for example in the group of biomarkers for PGP 9.5, GTPase activating proteins (among which GAP-43), beta-Ill tubulin, the vesicular glutamate transporter (VGLUT1), the tyrosine receptor kinase B and C (Trk B, C), brain-derived neurotrophic factor (BDNF), neurotrophin 3 (NT3), Calretinin, and said targeting agent can be retinoic acid.

### Composition

Also herein described is a pharmaceutical composition comprising nanoparticles and/or aggregates of nanoparticles such as herein above described, and a pharmaceutically acceptable carrier, vehicle, or support.

The said pharmaceutical composition is suitable for use in the treatment of neuropathic pain as described herein above.

### Technical Effect:

The therapeutic efficacy of the claimed nanoparticles is illustrated by the results (detailed in the Examples section), of experiments using animal models of neuropathic pain, namely a CIPN model (Examples 5 and 6) and a diabetes-induced neuropathic pain model (Example 7).

In Example 5, several embodiments of the claimed nanoparticles were demonstrated in the rats such as a long-lasting effect (at least 11 days) on pain relief due to CIPN after a single intraplantar injection. In the experiments, the animals were not evaluable beyond 11 days.

Thus, according to one embodiment of the invention, the claimed nanoparticles may be used to treat CIPN.

In Examples 6 and 7, a single intraplantar injection of hafnium oxide nanoparticles having a negatively charged biocompatible coating (Example 3) produced a long-lasting effect on pain relief (at least 7 days for the diabetes-induced NP model, and at least 11 days for the CIPN model). By contrast, the positive control, Pregabalin, had to be injected every 2 days, 2 hours before the Von Frey assessment to achieve constant pain relief during the testing period. Furthermore, a dose-effect (i.e., increasing doses of nanoparticles administered had an increased pain reducing effect) was clearly observed in the experiment of Example 6.

Thus, according to one embodiment of the invention, the claimed nanoparticles may be used to treat diabetes-induced neuropathic pain.

According to one embodiment of the invention increasing concentrations of nanoparticles leads to increased therapeutic efficacy.

The two animal models used to demonstrate the efficacy of the present nanoparticles are recognized as clinically relevant.

Based on the in vivo data from two different animal models, presented herein, the inventors believe the inventive nanoparticle composition may be used to treat all neuropathic pain conditions, preferably, peripheral neuropathic pain conditions.

Thus, the inventors have shown that the claimed nanoparticles may be used as a local treatment of peripheral neuropathic pain for a long-lasting pain relief. The local administration is advantageous because the negative side effects of a systemic treatment as detailed above are avoided. Furthermore, because the nanoparticles are administered infrequently compared to other locally administered neuropathic pain treatments, patient compliance is increased.

Other aspects and advantages of the invention will become apparent in the following examples, which are given for purposes of illustration and not by way of limitation.

### EXAMPLES

### Example 1. Nanoparticle Prepared from a Conductor Material: Synthesis of Gold Nanoparticles Coated with a Biocompatible Coating Presenting a Negative Surface Charge

Gold nanoparticles were synthesized by reducing a gold chloride salt (HAuCl₄) with a capping agent (sodium citrate) (protocol was adapted from G. Frens, (1973) Nature Physical Science 241, 21). In a typical experiment, HAuCl₄ solution was heated to boiling. Subsequently, sodium citrate solution was added. The resulting solution boiled for an additional period of 5 minutes then filtered. The gold concentration in suspension was determined by a UV-visible spectroscopy at 530 nm.

The resulting nanoparticles were coated with a biocompatible surface coating, meso-2, 3-dimercaptosuccinic acid (DMSA). A sufficient amount of DMSA was added to the nanoparticles suspension to reach at least half a monolayer coverage (2.5 molecules/nm²) on the surface.

The hydrodynamic diameter (measure in intensity) was determined by Dynamic Light Scattering (DLS) with a Nano-Zetasizer (Malvern) at a scattering angle of 1730 with a laser emitting at 633 nm, by diluting the nanoparticles suspension in water (final concentration 0.1 g/L). The hydrodynamic diameter of the so obtained nanoparticles in suspension was equal to 81 nm, with a polydispersity index (dispersion of the nanoparticles population in size) of 0.49.

The zeta potential was determined by measuring the electrophoretic mobility of the nanoparticles (Nano-Zetasizer, Malvern), by diluting the nanoparticles suspension in a NaCl solution at 1 mM at pH 7 (final concentration 0.1 g/L). The zeta potential at pH 7 was found equal to −32 mV.

The gold concentration was determined using Inductively Coupled Plasma-Optical Emission Spectrometry (ICP-OES).

### Example 2. Nanoparticle Prepared from an Insulator Material Having a Low Relative Dielectric Constant Equal to or Below 100: Synthesis of Zirconium Oxide Nanoparticles Coated with a Biocompatible Coating Presenting a Negative Surface Charge

Zirconium oxide (ZrO₂) nanoparticles were synthesized by precipitation of zirconium chloride (ZrCl₄) with tetramethyl ammonium hydroxide (TMAOH) at a basic pH. The resulting suspension was autoclaved at a temperature above 110° C., and after cooling, washed with deionized water, acidified and filtered (0.22 μm PES membrane filter). The nanoparticles (ZrO₂) concentration was determined as dry mass concentration

Coating was performed as follows. A sufficient mass of sodium hexametaphosphate was added to the nanoparticles suspension to reach at least half a monolayer coverage (2.5 molecules/nm²) on the surface.

The nanoparticles hydrodynamic diameter was measured by DLS to be 66 nm, with a polydispersity index (dispersion of the nanoparticles population in size) of 0.10.

The zeta potential was determined at pH 7 to be −40 mV.

The zirconium concentration was determined using Inductively Coupled Plasma-Optical Emission Spectrometry (ICP-OES).

### Example 3. Nanoparticle Prepared from an Insulator Material Having a Low Relative Dielectric Constant Equal to or Below 100: Synthesis of Hafnium Oxide Nanoparticles Coated with a Biocompatible Coating Presenting a Negative Surface Charge

Hafnium oxide (HfO₂) nanoparticles were synthesized by precipitation of Hafnium Chloride (HfCl₄) with tetramethyl ammonium hydroxide (TMAOH) at a basic pH. The resulting suspension was autoclaved above 110° C., cooled, washed with water and acidified.

Surface functionalization was performed using sodium hexametaphosphate as for Example 2.

The hydrodynamic diameter was measured by DLS to be 73 nm, with a polydispersity index (dispersion of the nanoparticles population in size) of 0.09. The zeta potential was determined as equal to −39 mV at pH 7.

The hafnium concentration was determined using Inductively Coupled Plasma-Optical Emission Spectrometry (ICP-OES).

### Example 4. Nanoparticle Prepared from an Insulator Material Having a Low Relative Dielectric Constant Equal to or Below 100: Synthesis of Hafnium Oxide Nanoparticles Coated with a Biocompatible Coating Presenting a Neutral Surface Charge

Hafnium oxide (HfO₂) nanoparticles were synthesized by precipitation of Hafnium Chloride (HfCl₄) with tetramethyl ammonium hydroxide (TMAOH) at a basic pH. The resulting suspension was autoclaved above 110° C., cooled, washed with deionized water and acidified.

A biocompatible coating was performed using silane-poly(ethylene) glycol 2 kDa (“Si-PEG 2 kDa”). A sufficient mass of Si-PEG 2 kDa was added to the nanoparticles suspension to reach at least half a monolayer coverage (2.5 molecules/nm²) on the surface.

The nanoparticles hydrodynamic diameter was found equal to 81 nm, with a polydispersity index (dispersion of the nanoparticles population in size) of 0.15.

The zeta potential was determined to be equal to −5 mV at pH7.

The hafnium concentration was determined using Inductively Coupled Plasma-Optical Emission Spectrometry (ICP-OES).

### Example 5: Efficacy of a Single Administration of Nanoparticles for the Symptomatic Treatment of Mechanical Allodynia in a Chemotherapy-Induced Peripheral Neuropathy Model in Rats (Oxaliplatin)

The aim of this study was to investigate the effect of a single intraplantar treatment with Nanoparticles from Example 1 (NP1B) at the dose of 11 g/L, Nanoparticles from Example 2 (NP2B) at 53 g/L, Nanoparticles from Example 3 (NP20B) at 57 g/L, and Nanoparticles from Example 4 (NP20A) at 59 g/L, each in 20 μL of injection volume, on the mechanical allodynia (as assessed using von Frey) that occurs following systemic administration of oxaliplatin in the rat.

### STUDY Design

The experimental design of the CIPN model is shown in Table 1.

TABLE 1
Experimental design of CIPN study from Example 5

| | | Single dose oxaliplatin intraperitoneally | | |
|---|---|---|---|---|
| | | (i.p.), single dose nanoparticle via | | |
| | | intraplantar administration (i.pl.) | | |
| Dosing Regimen | | Treatment | Treatment | No. of |
| Day | Test Article | dose/volume | Route | Animals |
| 0 | Administration of Oxaliplatin 10 | | | 72 total |
| | mg/kg i.p. to each group | | | |
| 9 | Group A: Saline | 20 μL injection | i.pl. | 12 |
| 9 | Group B: | 11 g/L/20 μL injection | i.pl. | 12 |
| | Nanoparticles | | | |
| | from Example 1 | | | |
| | (NP1B) | | | |
| 9 | Group C: | 53 g/L/20 μL injection | i.pl. | 12 |
| | Nanoparticles | | | |
| | from Example 2 | | | |
| | (NP2B) | | | |
| 9 | Group D: | 57 g/L/20 μL injection | i.pl. | 12 |
| | Nanoparticles | | | |
| | from Example 3 | | | |
| | (NP20B) | | | |
| 9 | Group E: | 59 g/L/20 μL injection | i.pl. | 12 |
| | Nanoparticles | | | |
| | from Example 4 | | | |
| | (NP20A) | | | |
| 9 | Group F: | 30 mg/kg | per os (p. o.) | 12 |
| | Pregabalin | | | |

### Material and Methods

All experiments were performed according to the Ethical Guidelines of the International Association for the Study of Pain (Zimmerman, 1983).

Experimental subjects: Seventy-two (72) adult male Sprague-Dawley rats were used for this study. The animals were housed in intra-ventilated cages in groups of four in a controlled environment of constant temperature and moisture (temperature: 21±1° C., light:dark cycle of 12:12 hours), with food and water available ad libitum. Animals were allowed to recover from transportation for at least one week before commencing experiments.

Static mechanical (tactile) allodynia evaluation: Measurement of paw withdrawal threshold (PWT) was achieved using calibrated (force; g) von-Frey monofilaments (Touch-Test Sensory Evaluator; Scientific Marketing Associates) applied to the plantar surface of the hind paw. Withdrawal threshold was determined by increasing and decreasing stimulus intensity and estimated using Dixon's up-down method.

The animals were placed on an elevated mesh bottom platform with a 0.5 cm² grid to provide access to the ventral side of the hind paws. An inverted plexiglass container was placed on top of each rat and testing was performed after an initial 15-20-minute acclimatisation/habituation period. The von Frey filaments were placed perpendicular to the plantar surface of the hind paw, from below the mesh floor. The monofilaments were held at the position for approximately 5 seconds with enough force to cause a slight bend of the filament. Only sharp withdrawal responses (or flinching) within up to five seconds from the stimulus were considered to represent a positive response.

### Study Schedule:

Baseline readings for latency to withdrawal (sec) were assessed on three consecutive days (Day −3, Day −2 and Day −1). The mean of Day −2 and Day −1 was considered the baseline prior to oxaliplatin dosing, and Day 0 (DO) the day of oxaliplatin injection. Neuropathic baseline recordings are taken on Day 5 (D5) and Day 6 (D6) after oxaliplatin.

Nanoparticles treated animals were injected intraplantar with a microneedles device into the left paw on D7 post-oxaliplatin. One single injection of nanoparticles was performed throughout the experiment. Pregabalin-treated animals (positive control) were dosed per os (p.o.) and tested 2 hr post dose. Nanoparticles treated animals were also tested 2 hr post dose on D7. Pregabalin was administered each day of von Frey testing.

Mechanical allodynia was subsequently re-assessed 2-3 times per week following injection, out to D26 post-oxaliplatin. Test days (post-oxaliplatin) were D7 (day of test compound dosing), D10, D14, D18, D22 and D26.

### Data Analysis

A two-way repeated measures ANOVA with ‘treatment’ as a between-subjects effect, and ‘day’ as a within-subjects effect was used (using GraphPad Prism version 9.0 for Windows, GraphPad Software, San Diego California USA), followed by Dunnett's multiple comparisons test. Data is presented as mean±SEM.

### Results

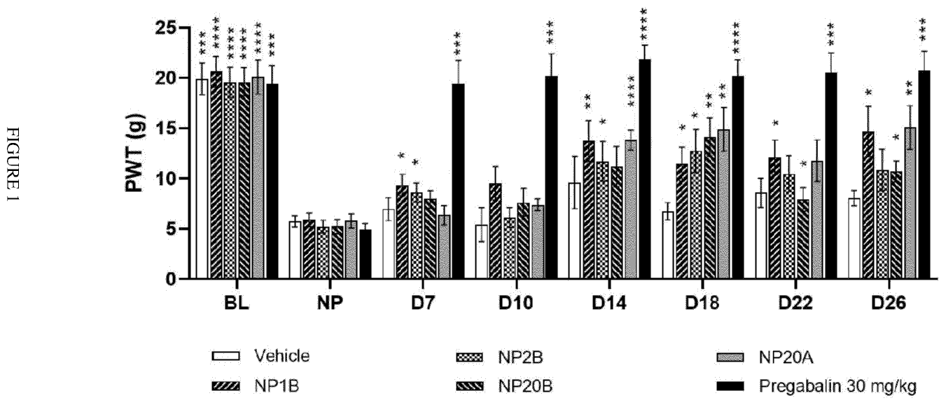

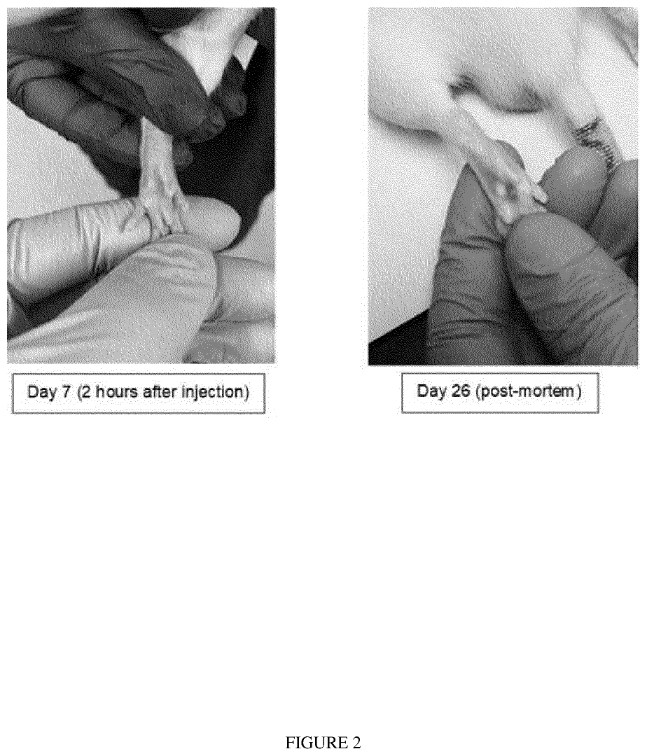

The values of paw withdrawal thresholds (PWTs) in rats after oxaliplatin-induced mechanical allodynia are shown in FIG. 1. FIG. 2 shows pictures of rat's paw just after the intraplantar administration of Nanoparticle from Example 1 (DO) and just after rat's sacrifice (D26).

### Conclusions

Administration of oxaliplatin induced consistent mechanical allodynic response as shown by paw withdrawal threshold (PWT) levels (FIG. 1). Systemic Pregabalin treatment, as a positive control, rescued the allodynic response and recovered the PWT to the healthy baseline levels.

Nanoparticles from Example 1 (NP1B) shows a significant increase in PWT when compared to its neuropathic baseline (FIG. 1), especially on D14 and onwards. Nanoparticles from Example 2 (NP2B) showed some increase when compared to its neuropathic baseline values (at D7, D14 and D18). Nanoparticles from Example 3 (NP20B) was especially effective on D18 and onwards, with a significant difference from its neuropathic baseline. Nanoparticles from Example 4 (NP20A) was consistently the most effective nanoparticle compound in reversing the mechanical allodynia from D14 and onwards, with a significant increase in PWT values from its neuropathic baseline. FIG. 2 shows that Nanoparticles from Example 1 stays where they have been injected with minimal elimination at least during 26 days.

### Example 6: Efficacy of a Single Administration of Nanoparticles from Example 3 for the Symptomatic Treatment of Mechanical Allodynia in a Chemotherapy-Induced Peripheral Neuropathy Model in Rats (Oxaliplatin): Dose Effect

The aim of this study was to investigate the effect of a single intraplantar administration of Nanoparticles from Example 3, at different doses, on mechanical allodynia in the rat oxaliplatin model of chemotherapy-induced neuropathic pain.

### Study Design

The experimental design of the CIPN model study is shown in Table 2.

Experimental design of CIPN study from Example 6

| | | Single dose of Oxaliplatin i.p., single | | | | |
|---|---|---|---|---|---|---|
| Dosing Regimen | | dose of nanoparticles intraplantar. | | | | |
| | Test | Pre- | | | | No of |
| Day | Article | treatment | Route | Treatment | Route | Animals |
| 0 | Administer Oxaliplatin 10 mg/kg i.pl. or Vehicle | | | | | 60 for |
| | according to group | | | | | behaviour |
| 9 | Group A: | Vehicle at | i.pl. | Vehicle | intraplantar | 12 |
| | Vehicle (no | 5 mL/kg | | | | |
| | oxaliplatin) | | | | | |
| 9 | Group B: | Oxaliplatin | i.pl. | Nanoparticles | intraplantar | 12 |
| | Nanoparticles | | | 61 g/L/20 μl | | |
| | from Example 3 | | | injection | | |
| 9 | Group C: | Oxaliplatin | i.pl. | Nanoparticles | intraplantar | 12 |
| | Nanoparticles | | | 18 g/L/20 μl | | |
| | from Example 3 | | | injection | | |
| 9 | Group D: | Oxaliplatin | i.pl. | Nanoparticles | intraplantar | 12 |
| | Nanoparticles | | | 18 g/L/10 μl | | |
| | from Example 3 | | | injection | | |
| 9 | Group E: | Oxaliplatin | i.pl. | 30 mg/kg | p.o. | 12 |
| | Pregabalin | | | | | |

Note:
In this study, each animal constitutes its own Neuropathic control, since ‘treatment’ has been injected into the left paw via intraplantar injection, whereas ‘Vehicle’ has been injected into the right paw via intraplantar injection, so that paw withdrawal threshold (PWT) measurements on the right paw constitute the ‘Neuropathic Control group’. A ‘Vehicle/Vehicle’ group (‘healthy animals’) has also been included.

### Material and Methods

All experiments were performed according to the Ethical Guidelines of the International Association for the Study of Pain (Zimmerman, 1983).

Experimental subjects: Sixty (60) adult male Sprague-Dawley rats (180-210 g) were used for this study. The animals were housed in intra-ventilated cages in groups of four in a controlled environment of constant temperature and moisture (temperature: 21±1° C., light: dark cycle of 12:12 hours) with food and water available ad libitum. Animals were allowed to recover from transportation for at least one week before commencing experiments.

Static mechanical (tactile) allodynia: Measurement of withdrawal threshold was achieved using calibrated (force; g) von-Frey monofilaments (Touch-Test Sensory Evaluator; Scientific Marketing Associates) applied to the plantar surface of the hind paw. Withdrawal threshold was determined by increasing and decreasing stimulus intensity and estimated using Dixon's up-down method.

The animals were placed on an elevated mesh bottom platform with a 0.5 cm² grid to provide access to the ventral side of the hind paws. An inverted plexiglass container was placed on top of each rat and testing was performed after an initial 15-20-minute acclimatisation/habituation period. The von-Frey filaments were placed perpendicular to the plantar surface of the hind paw, from below the mesh floor. The monofilaments were held at the position for approximately 4 s with enough force to cause a slight bend of the filament. Only sharp withdrawal responses (or flinching) within up to five seconds from the stimulus were considered to represent a positive response.

Study schedule: Baseline readings for latency to withdrawal (sec) were assessed on three consecutive days (Day −3, Day −2 and Day −1). The mean of Day −2 and Day −1 was considered the baseline prior to oxaliplatin dosing, and Day 0 (DO) the day of oxaliplatin injection. Neuropathic baseline recordings were taken on day 5 (D5) after oxaliplatin.

Nanoparticles treated animals were injected intraplantar with a microneedles device into one paw on Day 6 (D6) post-oxaliplatin (the opposite paw was used as a control, by injecting with a microneedles device the same volume of Vehicle—Saline). One single injection of nanoparticles was performed throughout the experiment. Pregabalin-treated animals (positive control) were dosed per os (p.o.) and tested for mechanical allodynia 2 hr post dose. Nanoparticles treated animals were also tested 2 hrs post dose on D6. Pregabalin was administered each day of von Frey testing.

Mechanical allodynia was subsequently re-assessed 2-3 times per week following injection out to Day 24 (D24) post-oxaliplatin. Test days (post-oxaliplatin) were D6 (day of test compound dosing), D9, D13, D16, D20 and D24.

### Data Analysis

A two-way repeated measures ANOVA with ‘treatment’ as a between-subjects effect, and ‘day’ as a within-subjects effect was used (using GraphPad Prism version 9.0 for Windows, GraphPad Software, San Diego California USA), followed by Dunnett's multiple comparisons test. Data is presented as mean±SEM.

### Results

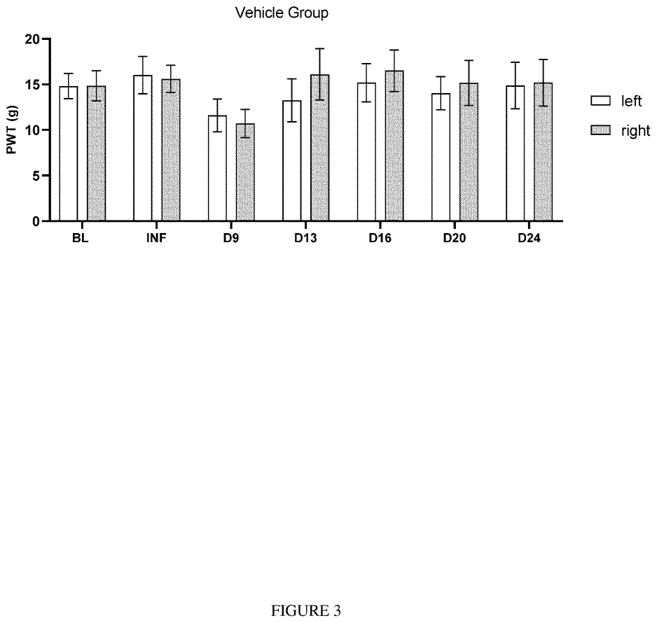

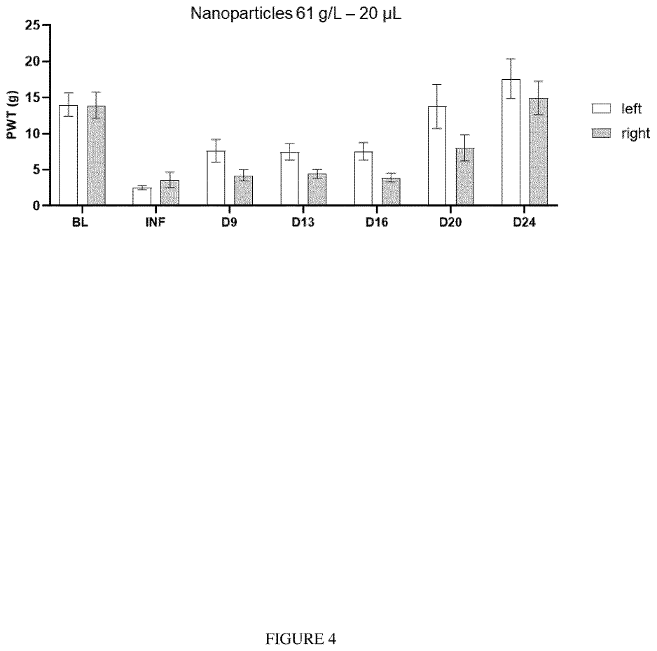

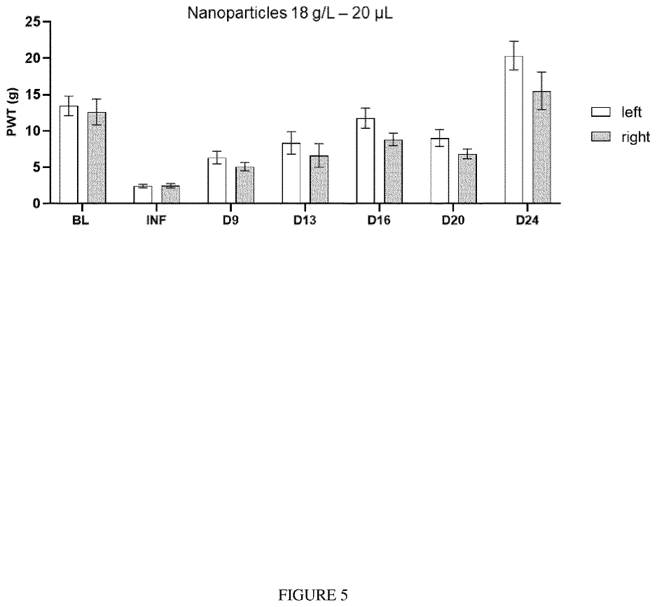

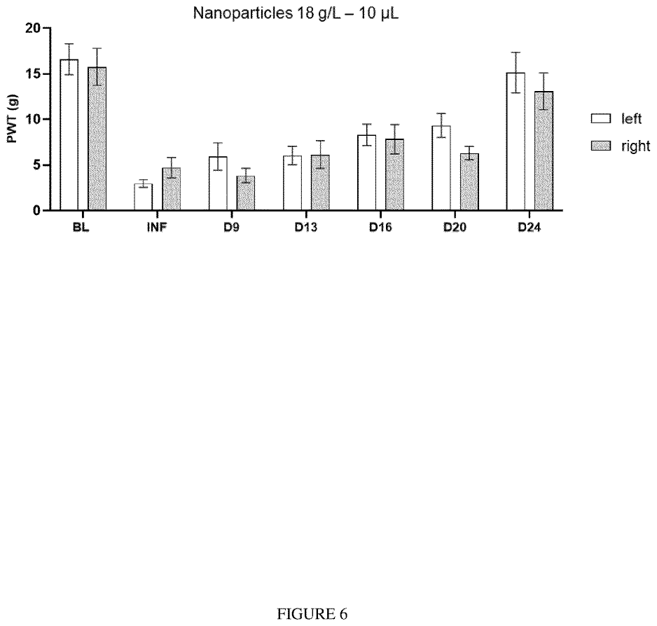

The values of paw withdrawal thresholds (PWTs) in rats after oxaliplatin-induced mechanical allodynia are shown in FIGS. 3, 4, 5, 6 and 7, and significance of results is evaluated in Tables 3, 4, 5, 6 and 7, below.

TABLE 3
CIPN study from Example 6: Summary of mixed-effects
analysis for ‘Vehicle/Vehicle’ Group (Dunnett's
multiple comparisons test)

| Source of Variation | P value | P value summary |
|---|---|---|
| Time × Group | 0.9418 | ns |
| Time | 0.1317 | ns |
| Group (left vs. right paw) | 0.7654 | ns |
| Subject | <0.0001 | **** |

(“ns”: non-significant;
* P value < 0.05;
** P value < 0.01;
*** P value < 0.001;
**** P value < 0.0001).

TABLE 4
CIPN study from Example 6: Summary of mixed-effects
analysis for ‘Nanoparticles from Example 3 - 61
g/L-20 μL’ Group (Dunnett's multiple comparisons test)

| Source of Variation | P value | P value summary |
|---|---|---|
| Time × Group | 0.4155 | ns |
| Time | <0.0001 | **** |
| Group (left vs. right paw) | 0.0326 | * |
| Subject | 0.0612 | ns |

(“ns”: non-significant;
* P value < 0.05;
** P value < 0.01;
*** P value < 0.001;
**** P value < 0.0001).

TABLE 5
CIPN study from Example 6: Summary of mixed-effects
analysis for ‘Nanoparticles from Example 3 - 18
g/L-20 μL’ Group (Dunnett's multiple comparisons test)

| Source of Variation | P value | P value summary |
|---|---|---|
| Time × Group | 0.5784 | ns |
| Time | <0.0001 | **** |
| Group (left vs. right paw) | 0.0678 | ns |
| Subject | 0.0058 | ** |

(“ns”: non-significant;
* P value < 0.05;
** P value < 0.01;
*** P value < 0.001;
**** P value < 0.0001).

TABLE 6
CIPN study from Example 6: Summary of mixed-effects
analysis for ‘Nanoparticles from Example 3 - 18
g/L-10 μL’ Group (Dunnett's multiple comparisons test)

| Source of Variation | P value | P value summary |
|---|---|---|
| Time × Group | 0.5997 | ns |
| Time | <0.0001 | **** |
| Group (left vs. right paw) | 0.4304 | ns |
| Subject | 0.0001 | *** |

(“ns”: non-significant;
* P value < 0.05;
** P value < 0.01;
*** P value < 0.001;
**** P value < 0.0001).

TABLE 7
CIPN study from Example 6: Summary of mixed-effects
analysis for ‘Pregabalin/Oxaliplatin’ Group
(Dunnett's multiple comparisons test)

| Source of Variation | P value | P value summary |
|---|---|---|
| Time × Group | 0.317 | ns |
| Time | <0.0001 | **** |
| Group (left vs. right paw) | 0.3211 | ns |
| Subject | 0.0003 | *** |

(“ns”: non-significant;
* P value < 0.05;
** P value < 0.01;
*** P value < 0.001;
**** P value < 0.0001).

### Conclusions

Administration of oxaliplatin induced consistent mechanical allodynic response on the days assessed until Day 20 post administration as shown by paw withdrawal threshold (PWT) levels in the right paw. As known from literature (W. H. Xiao, H. Zheng, and G. J. Bennett, Neuroscience. 2012, 203: 194-206: Characterization of oxaliplatin-induced chronic painful peripheral neuropathy in the rat and comparison to the neuropathy induced by paclitaxel) and as observed from the PWT of the untreated (right) paws from all groups that are at the level of the baseline (before neuropathy) at Day 24, the mechanical allodynic adverse effect of oxaliplatin starts to decrease after 4 weeks because of oxaliplatin washing. Systemic Pregabalin treatment worked as a positive control since, for both left and right paws, levels of PWT were not significantly different from the ‘Vehicle/Vehicle’ group (not neuropathic) on any of the test days.

Nanoparticles from Example 3 at a concentration of 61 g/L and at a volume of administration of 20 μL were responsible for reduced pain response on the treated (left) paw since data shows higher paw withdrawal threshold (PWT) values on the left paw when compared to the right paw, and since statistical analysis showed a significant effect of side (left paw versus right paw), with a p value <0.05 (p value=0.0326; cf. Table 4), whereas statistical analysis didn't show a significant effect of side for Nanoparticles from Example 3 at 18 g/L, at volumes of 20μL (p value=0.0678) and 10 μL (p value=0.4304) (cf. Table 5 and Table 6). This result indicates a minimal dose threshold for Nanoparticles from Example 3 to be significantly efficient in reducing mechanical allodynia induced by oxaliplatin-induced peripheral neuropathy in rats. A single injection of Nanoparticles from Example 3 at a concentration of 61 g/L (20 μL) induced pain relief from Day 9 to Day 20, meaning a pain relief lasting at least 11 days, whereas the positive control Pregabalin had to be injected every 2 days (2 hrs before the Von Frey assessment).

### Example 7: Efficacy of a Single Administration of Nanoparticles for the Symptomatic Treatment of Mechanical Allodynia in a Diabetes-Induced Peripheral Neuropathy Model in Rats (Streptozotocin)

A major cause of neuropathic pain, observed in the clinic, arises from diabetes. In rodents, this is mimicked by streptozotocin (STZ) injection to induce diabetes. STZ is an antibiotic that interferes with the production of insulin by destroying the pancreatic islet cells and, therefore, rats develop diabetes and diabetes-induced neuropathic pain. The rat STZ model of diabetes has successfully been used to model Type-1 diabetes-induced neuropathic pain and provided key decision-making allodynia efficacy data for the development of pregabalin, one of very few treatments that has successfully translated preclinical neuropathic pain results into clinical success.

Diabetes is confirmed by testing blood glucose levels. The neuropathic pain symptoms, e.g., mechanical allodynia (measured using von-Frey filaments), usually develop between two to six weeks following STZ injection.

The aim of the study is to investigate the effect of a single intraplantar treatment with Nanoparticles from Example 3 at the doses of 117 g/L in 20 μL of injection volume, 58 g/L in 20 μL of injection volume, and 18 g/L in 10 μL of injection volume on the mechanical allodynia (as assessed using von Frey) that occurs following systemic administration of Streptozocin in the rat.

### Study Design

The experimental design of the STZ-induced diabetic neuropathy model is shown in Table 8.

TABLE 8
Experimental design of STZ-induced diabetic NP study (Example 7)

| | | Dose | Day of | | | | |
|---|---|---|---|---|---|---|---|
| | STZ | Level | Test | Test Article | | | |
| Group | treatment | and | Article | (left hind | Nanoparticles | Dose vol/ | No. of |
| ID | (Day 0) | route | Dose | paw) | concentration | route | Animals |
| A | Vehicle | 10 mL/kg | D14 | Vehicle | n/a | 20 μl | 6 |
| | | IP | | | | intraplantar | |
| B | Streptozocin | 55 | D14 | Vehicle | n/a | 20 μl | 12 |
| | | mg/kg | | | | intraplantar | |
| | | IP | | | | | |
| C | Streptozocin | 55 | D14 | Nanoparticles | 117 g/L | 20 μl | 12 |
| | | mg/kg | | from | | intraplantar | |
| | | IP | | Example 3 | | | |
| D | Streptozocin | 55 | D14 | Nanoparticles | 58 g/L | 20 μl | 12 |
| | | mg/kg | | from | | intraplantar | |
| | | IP | | Example 3 | | | |
| E | Streptozocin | 55 | D14 | Nanoparticles | 18 g/L | 10 μl | 12 |
| | | mg/kg | | from | | intraplantar | |
| | | IP | | Example 3 | | | |
| F | Streptozocin | 55 | D14 | Pregabalin | 30 mg/kg | p.o. | 12 |
| | | mg/kg | | | | | |
| | | IP | | | | | |

### Material and Methods

All experiments were performed according to the Ethical Guidelines of the International Association for the Study of Pain (Zimmerman, 1983). All behavioural tests were approved by the United Kingdom Home Office Animals (Scientific Procedures) Act 1986.

Experimental subjects: Sixty-six (66) adult male Sprague-Dawley rats (180-210 g) were used for this study. The animals were housed in intra-ventilated cages in groups of two in a controlled environment of constant temperature and moisture (temperature: 21±1° C., light: dark cycle of 12:12 hours) with food and water available ad libitum. Animals were allowed to recover from transportation for at least one week before commencing experiments.

Streptozotocin-induced model of diabetes: Animals were injected with STZ (Day 0) (55 mg/kg, i.p., 10 mL/kg, single injection) or its Vehicle solution (20 mM citrate buffer, i.p., 10 mL/kg, single injection). For 48 hours post STZ injection, animals received 2% sucrose in the drinking water (a choice of 1 bottle of water and 1 bottle of 2% sucrose was provided) to manage the transient hypoglycaemic phase typically occurring between 4-8 hours post injection of STZ. Following STZ injection, all animals were switched to a slightly higher protein diet (Labdiet 5LF5; 22% protein).

Body weights monitored daily throughout the study. STZ rats normally lose between 5-10% of their baseline body weight and usually begin to gain body weight back to baseline level over 4 weeks. Observable STZ-induced toxicity (<5% of the total n number) took place 3-4 days post injection. Typical symptoms in diabetic animals include polydipsia, polyuria, glycosuria and hyperglycaemia. To minimise suffering the diabetic animals were provided with extra drinking water because they were polydipsic and their home cages were changed more frequently (daily) due to polyuria. Rats were cleaned out daily following i.p. STZ injection and after behavioural testing.

Confirmation of hyperglycaemia: On Day 7 (D7) one drop of tail vein blood was obtained by a needle prick on the conscious rats. The blood glucose levels were measured in one drop of blood using an Exactive Vital (MictoTech Medical) blood glucose monitor. Only STZ injected rats with blood glucose concentrations above 16 mmol/L were considered diabetic and included in this study (expected >90%).

Static mechanical (tactile) allodynia: Measurement of withdrawal threshold was achieved using calibrated (force; g) von-Frey monofilaments (Touch-Test Sensory Evaluator; Scientific Marketing Associates) applied to the plantar surface of the hind paw. Withdrawal threshold was determined by increasing and decreasing stimulus intensity and estimated using Dixon's up-down method (Dixon, 1980; Chaplan et al., 1994).

The animals were placed on an elevated mesh bottom platform with a 0.5 cm² grid to provide access to the ventral side of the hind paws. An inverted plexiglass container was placed on top of each rat and testing was performed after an initial 15-20-minute acclimatisation/habituation period. The von-Frey filaments were placed perpendicular to the plantar surface of the hind paw, from below the mesh floor. The monofilaments were held at the position for approximately 4 s with enough force to cause a slight bend of the filament. Only sharp withdrawal responses (or flinching) within up to five seconds from the stimulus were considered to represent a positive response.

Study schedule: Nanoparticle treated animals are dosed locally as an intraplantar injection with a microneedles device into a single paw. Pregabalin is dosed per os 2 hr prior to each Von Frey test. Nanoparticles are only dosed once, on Day 14 (D14). Control groups (‘Vehicle/Vehicle’ and ‘STZ/Vehicle’) are injected once (on D14) with Saline in a single paw, with microneedles.

Von Frey readings are taken at baseline (pre-STZ), at D9 and at D11 post-STZ (neuropathic/allodynia baseline) and 2 hours post dose on D14. Von-Frey readings are repeated twice per week up to 3 weeks post-STZ (1 week post nanoparticles dosing).

### Data Analysis

A two-way repeated measures ANOVA with ‘treatment’ as a between-subjects effect, and ‘day’ as a within-subjects effect was used (using GraphPad Prism version 9.0 for Windows, GraphPad Software, San Diego California USA), followed by Dunnett's multiple comparisons test. Data is presented as mean±SEM.

### Results

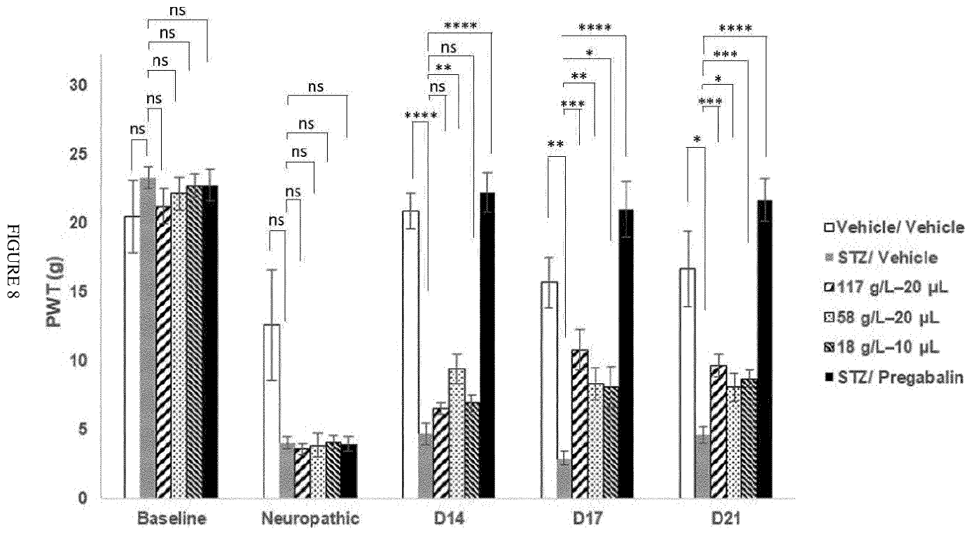

The values of paw withdrawal thresholds (PWTs) in rats after STZ-induced mechanical allodynia are shown in FIG. 8 and significance of results is evaluated in Table 9, below.

TABLE 9
STZ-induced diabetic NP study (Example 7): Summary of mixed-
effects analysis (Dunnett's multiple comparisons test)

| Fixed effects | | P value | Statistically significant |
|---|---|---|---|
| (type III) | P value | summary | (P < 0.05) |
| Time | <0.0001 | **** | Yes |
| Group | <0.0001 | **** | Yes |
| Time × Group | <0.0001 | **** | Yes |

(“ns”: non-significant;
* P value < 0.05;
** P value < 0.01;
*** P value < 0.001;
**** P value < 0.0001)

### Conclusions

Glucose blood levels: All STZ-treated animals showed increased blood glucose at Day 7 (D7) with levels higher than 16 mmol/L (data not shown). This increase was statistically significant when comparing mean of different groups at Day 0 (DO) (day of STZ administration and Day 7).

It is to be noted that the model has been run until D38, but due to abnormal behaviors caused in animals by STZ′ side effects (diarrhoea, increased behavioural signs of stress response during handling), animals from the naïve group, though not showing any clinical symptoms as expected, also showed some behavioural signs of stress, such as restlessness while being handled, which results in non-interpretable results from D24 to D38.

Mechanical allodynia: STZ treated animals were scored for neuropathic mechanical allodynia on days 9 (D9) and 11 (D11). All animals showed increased levels of mechanical allodynia.

Systemic Pregabalin treatment worked as a positive control. PWT levels were significantly different from the ‘STZ/Vehicle’ group on all days tested.

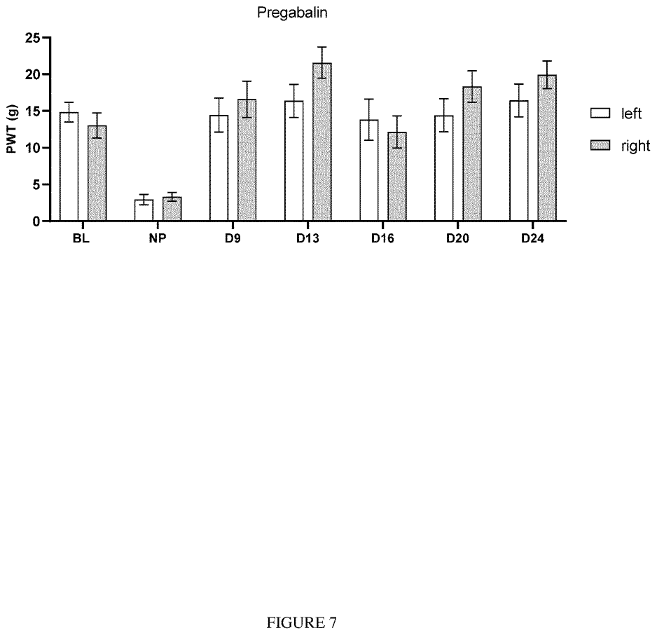

Treatment with nanoparticles at different doses was responsible for statistically significant higher PWT levels on D14, D17 and D21, for the group treated with a concentration of 58 g/L when compared to ‘STZ/Vehicle’ Group (FIG. 7). It is to be noted that the lowest dose of Nanoparticles from Example 3, 18 g/L-10 μL, gave a significantly higher PWT level from the ‘STZ/Vehicle’ group with a p value <0.001 on D21, meaning that this dose is sufficient to have efficacy of Nanoparticles from Example 3 on the symptomatic treatment of mechanical allodynia in a diabetes-induced neuropathic pain model in rats.

1-15. (canceled)

16. A method of treating peripheral neuropathic pain in a subject comprising administering nanoparticles or aggregates thereof to a subject having peripheral neuropathic pain and wherein the nanoparticles or nanoparticle aggregates are not exposed to an electric field nor to any other external activation source, wherein i) the nanoparticle material is a metallic conductor selected from Ir, Pd, Pt, Au and any mixture thereof; an organic material having contiguous sp² hybridized carbon centers in its structure selected from poly(3,4-ethylenedioxythiophene) (PEDOT), poly(styrene sulfonate) (PSS) and a mixture thereof; or an insulator material with a dielectric constant εᵢⱼₖ equal to or below 100 which is a metal oxide or a mixed metal oxide wherein the metal is Zr and/or Re, ii) when the nanoparticle material is a metallic conductor, the nanoparticle or nanoparticle aggregate has a median population core size of at least 45 nm, and iii) the core of the nanoparticle or nanoparticles' aggregate is coated with a biocompatible coating providing a neutral or a negative surface charge when measured in a solution of water having an electrolyte concentration between 0.001 and 0.2 M, a concentration of the nanoparticles or nanoparticle aggregates material between 0.01 and 10 g/L and a pH between 6 and 8.

17. The method according to claim 16, wherein the subject to be treated suffers from trigeminal neuralgia, neuropathic pain following peripheral nerve injury, post-traumatic neuropathic pain, post-surgical neuropathic pain or neuroma-induced neuropathic pain, painful polyneuropathy associated with diabetes, human immunodeficiency virus (HIV), Fabry disease, sodium channel gene mutation(s), autoimmune diseases like Guillain-Barre syndrome, vasculitis, chronic inflammatory demyelinating polyneuropathy, amyloidosis, nonfreezing cold injury, paraneoplastic syndrome or leprosy, chemotherapy-induced peripheral neuropathy (CIPN), post herpetic neuralgia, or painful radiculopathy.

18. The method according to claim 17, wherein the subject to be treated suffers from peripheral nerve injury, post-traumatic neuropathic pain, post-surgical neuropathic pain or neuroma-induced neuropathic pain, post herpetic neuralgia, painful polyneuropathy associated with diabetes, CIPN, or HIV.

19. The method according to claim 18, wherein the subject to be treated suffers from neuropathic pain following peripheral nerve injury, post-traumatic neuropathic pain, post-surgical neuropathic pain, or neuroma-induced neuropathic pain.

20. The method according to claim 18, wherein the subject to be treated suffers from painful polyneuropathy associated with diabetes or from CIPN.

21. The method according to claim 16, wherein the subject suffers from focal neuropathic pain.

22. The method according to claim 16, wherein the subject suffers from refractory and/or chronic neuropathic pain.

23. The method according to claim 16, wherein the nanoparticles or nanoparticle aggregates are administered by intradermal and/or intraepidermal injection.

24. The method according to claim 23, wherein the dose of nanoparticles or aggregates to be administered is at least 60 μg/cm² of skin surface injected.

25. The method according to claim 16, wherein the nanoparticle material is ZrO₂.

26. The method according to claim 16, wherein the nanoparticle material is selected from Ir, Au, Pd, Pt and any mixture thereof.

27. The method according to claim 25, wherein the patient suffers from diabetic neuropathy and the dose of nanoparticles or aggregates administered is at least 60 μg/cm² of skin surface to be injected.

28. The method according to claim 25, wherein the patient suffers from CIPN and the dose of nanoparticles or aggregates administered is at least 390 μg/cm² of skin surface to be injected.

29. The method according to claim 26, wherein the patient suffers from CIPN and the dose of nanoparticles or aggregates administered is at least 70 μg/cm² of skin surface to be injected.

30. The method according to claim 16, wherein the nanoparticles or aggregates thereof are administered using a dissolvable microneedle patch.
